(19) United States
(12) Patent Application Publication
(10) Pub. No.: US 2003/0109910 A1
(43) Pub. Date: Jun. 12, 2003

(54) Heating or cooling pad or glove with phase change material

(76) Inventors: Charles A. Lachenbruch, Summerville, SC (US); Richard I. Barnett, Georgetown, SC (US)

Correspondence Address: KATHLEEN M HARLESTON, THE HARLESTON LAW FIRM, 909 TALL PINE ROAD, MT PLEASANT, SC, 29464

(21) Appl. No.: 10/013,443

(22) Filed: Dec. 8, 2001

(51) Int. Cl. A61F 5/00; A61F 7/00; A61F 7/12

(52) U.S. Cl. 607/108; 607/111; 607/112; 604/114

(57) ABSTRACT

A heating or cooling pad for reducing pain or swelling, or increasing range of motion, includes: (a) phase change material having a melting point of between about 42 and 65 degrees Centigrade, or between about −10 and 6 degrees Centigrade; (b) a gel or viscous fluid carrier in which the phase change material is relatively evenly distributed; and (c) a fluid-impermeable, conformable envelope surrounding the phase change material and the carrier. A preferred embodiment herein includes: (a) a fluid-impermeable, flexible, conformable envelope; (b) a mixture of from about 10 to about 75 weight % of alkanes having a carbon chain length of between 12 and 14 or 22 and 30, and from about 25 to about 90 weight % of a gel or viscous fluid carrier in which the alkanes are substantially evenly distributed, the mixture being sealed within the envelope; and (c) at least one layer of insulation adjacent to a first side of the envelope. The envelope is preferably sandwiched between comfortable, conformable layers of insulation.

### BACKGROUND OF THE INVENTION

1. Technical Field

The present device is an unpowered glove, mitt, arm wrap, or pad containing phase change material with a melting point of between about 42 and 65 degrees Centigrade, or between about −10 and 6 degrees Centigrade, the phase change material being distributed in a gel or viscous fluid carrier, for sustained heating or cooling of a localized area of the body.

2. Background Information

Carpal tunnel syndrome, arthritis, and back pain are endemic in the American population of late. The former is common among workers who have been performing tasks with repeated finger, hand or wrist motions for years, particularly seamstresses, factory workers and secretaries. People with the second ailment, arthritis, in their hands suffer greatly from pain in their hands and wrists, particularly on awakening in the morning. Arthritis very seldom recedes, and more often intensifies over time. There is no cure for arthritis, only treatments, such as taking aspirin. Back problems cause widely differing pain levels. Back pain can range from mildly annoying to incapacitating, and many types of back pain do not respond to treatment. Back pain sufferers may have cracked vertebrae, spondylosis, degenerative joint disease, rheumatoid arthritis, or sprains, for example. Unfortunately, it is often difficult for physicians to pinpoint the source of back pain. For those whose ailment or discomfort intensifies, an operation may be necessary. Carpal tunnel syndrome, for example, is commonly treated by severing a ligament in the wrist.

Many physicians and patients recommend the application of heat to painful areas on the back or hands, as a treatment rather than a cure. Back pain sufferers, for example, often lay an electric heating pad on the troublesome area of their back. Sometimes, cool compresses are useful, for example, on the wrists or lower arms of those with carpal tunnel syndrome, involuntary tremors, or in the area of a sprain or swelling. There is a need for a handy device that will focus a comfortable and therapeutic level of heat or cooling on an affected area of the body for a sufficient period of time to bring at least temporary relief to sufferers.

The present invention is an unpowered, easy to use glove, mitt, wrist band, arm wrap, or the like, or a heating/cooling pad insert that can be warmed in a microwave oven or any suitable warming device, or cooled in a refrigerator or freezer, or by any other suitable means of cooling, and then worn over a person's hand and/or wrist, or placed on any area of the body. The term “glove” as used herein is meant to include similar type articles, such as a mitt, wrist band, or arm wrap. Cooling or warming of the skin is accomplished in the present invention by means of an envelope containing specific phase change materials (PCMs) distributed in a gel or viscous fluid carrier. The glove or pad insert (“article”) incorporates the envelope with the phase change materials so the article stays warm or cool longer and therefore temporarily relieves hand or wrist pain (caused by, e.g., arthritis, carpal tunnel syndrome, sprains), soreness and swelling (e.g., from athletics or bruising), or involuntary tremors. The use of PCMs also ensures that the heat or cold that is delivered to the skin at precisely selected temperatures that have been found to be therapeutic for the particular condition.

The present invention also includes a heating or cooling pad device with an insert that can be warmed in a microwave or the like, or cooled in a refrigerator or the like. The pad device is then placed over a sore back, bruised area, injury, etc. The heating/cooling pad contains phase change materials so the pad stays warm (or cool) longer and therefore relieves pain or soreness in the area for a longer period. Additionally, this heating or cooling is delivered to the tissue at a temperature controlled by the temperature of the phase change, which ensures both safe and effective therapeutic application of heat. The preferred embodiment is a PCM-based, low back heating and support device.

In operation, the pad insert, glove or other article can be placed in a freezer, refrigerator, or other cooling environment at a temperature cool enough to cause solidification of the majority of the included phase change material. Alternatively, the article can be microwaved or otherwise heated until the PCM in the article is suitably heated to the temperature of the phase change. The two distinct applications require that the articles be heated or cooled to two different temperature ranges. A visible temperature indicator is preferably included on the article so the wearer knows when to remove it from the heater or freezer. The glove or pad insert is removed from the refrigerator or heater and placed on the hand, wrist, and/or forearm, or in the pad device, to provide therapeutic heat to those areas over an extended period of time.

When warmed, the temperature of the article will warm to the particular temperature of phase change. The additional input of heat energy will cause further melting. It is believed that as long as some fraction of the PCM remains in the solid state, the temperature of the warming pad insert will be held constant at the temperature of phase change.

### BRIEF SUMMARY OF THE INVENTION

The present invention provides a heating or cooling pad for reducing pain or swelling, or increasing range of motion, including:

(a) phase change material having a melting point of between about 42 and 65 degrees Centigrade, or between about −10 and 6 degrees Centigrade;

(b) a gel or viscous fluid carrier in which the phase change material is substantially evenly distributed; and

(c) a fluid-impermeable, conformable envelope surrounding the phase change material and the carrier.

A preferred embodiment herein includes: (a) a fluid-impermeable, flexible, conformable envelope; (b) a mixture of from about 10 to about 75 weight % of alkanes having a carbon chain length of between 12 and 14 or 22 and 30, and from about 25 to about 90 weight % of a gel or viscous fluid carrier in which the alkanes are substantially evenly distributed, the mixture being sealed within the envelope; and (c) at least one layer of insulation adjacent to a first side of the envelope.

Phase change materials for use in a heating glove or heating pad of the present invention have a phase change such that temperatures of the skin area covered will be brought to between about 37° and 44° C. For a cooling article according to the present invention, the skin under the article is cooled to a narrow temperature range in the 15 to 27 degree Centigrade range. The normal core temperature (“normothermia”) of the human body is between about 36° and 38° C. Skin temperature typically ranges between about 31° C. and about 38° C.

### BRIEF DESCRIPTION OF THE SEVERAL VIEWS OF THE DRAWINGS

A more complete understanding of the invention and its advantages will be apparent from the following detailed description taken in conjunction with the accompanying drawings, wherein examples of the invention are shown, and wherein:

FIG. 1 shows a top perspective view of a glove according to the present invention, including a cutaway view of the interior of the glove;

FIG. 2 is a cross-sectional view of a portion of the glove of FIG. 1;

FIG. 3 is a bottom perspective view of a glove according to the present invention, showing a hand in the glove;

FIG. 4 shows a top perspective view of a mitt according to the present invention, showing a hand entering the mitt;

FIG. 5 is a cross-sectional view of the mitt of FIG. 4, taken at line 5-5;

FIG. 6 is a side elevational view of the mitt of FIG. 4;

FIG. 7 shows a side perspective view of a mitt according to FIG. 4, showing the outline of a hand in the mitt;

FIG. 8 shows a top perspective view of a glove according to the present invention, showing the outline of a hand in the glove;

FIG. 9 shows a side perspective view of an arm wrap according to the present invention;

FIG. 10 is a front perspective view of a lower back heating/cooling pad device according to the present invention;

FIG. 11 is a front perspective view of a lower back pad device according to the present invention, shown on a body;

FIG. 12 is a rear perspective view of a lower back pad device according to FIG. 11;

FIG. 13 is a front perspective view of a heating/cooling pad vest device according to the present invention, shown on a body;

FIG. 14 is a rear perspective view of a pad vest device according to FIG. 13; and

FIG. 15 is a cross-sectional view of a pad insert according to the present invention.

### DETAILED DESCRIPTION OF THE INVENTION

In the following description, like reference characters designate like or corresponding parts throughout the several views. Also, in the following description, it is to be understood that such terms as “front,” “back,” “within,” and the like are words of convenience and are not to be construed as limiting terms. Referring in more detail to the drawings, the invention will now be described.

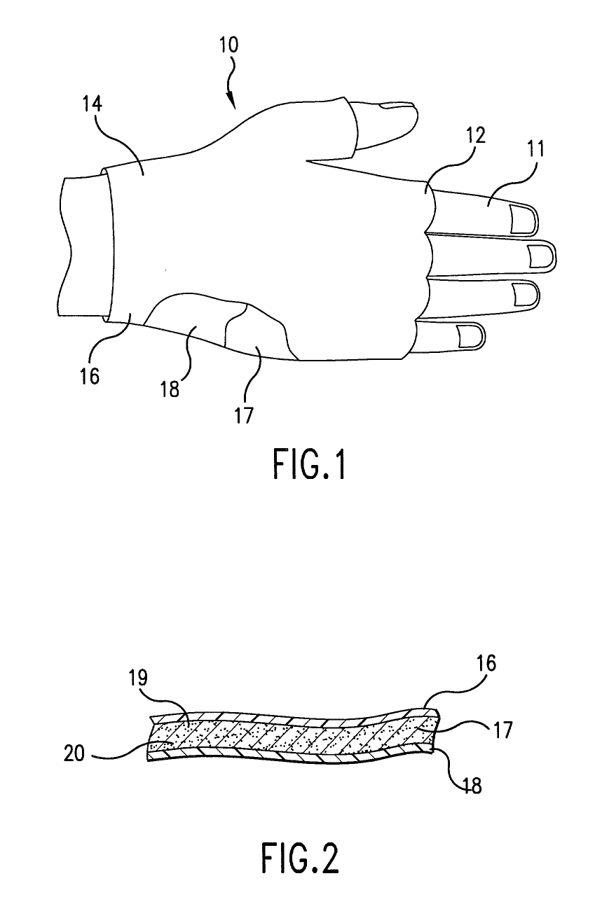

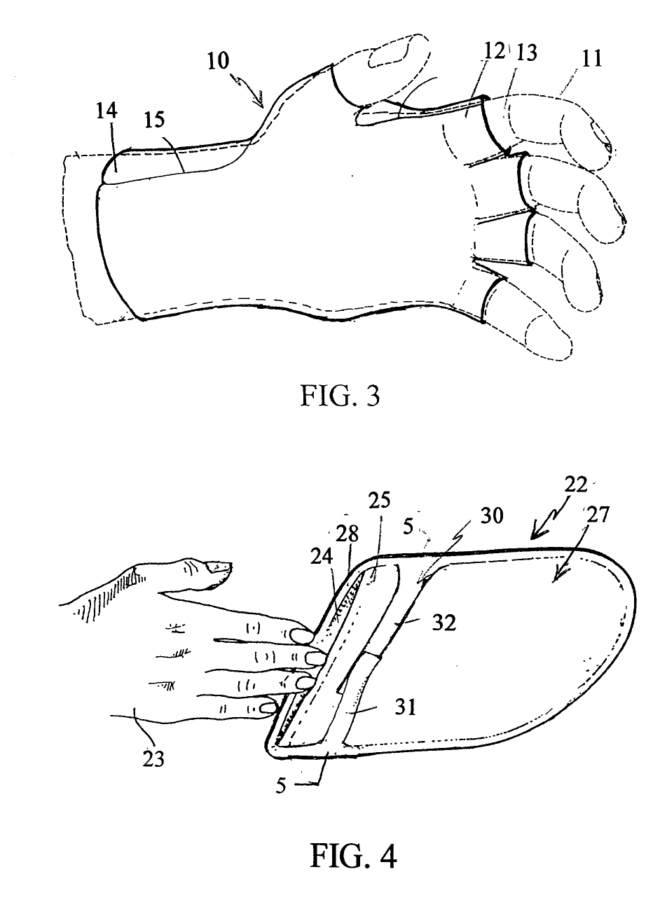

Turning first to FIG. 1, a glove, generally referred to as 10, according to the present invention includes a cutaway portion for purposes of illustration. The glove embodiment illustrated in FIG. 1 does not have fingertips, so the upper portions of the user's fingers 11 are left free for manipulating items. As shown in FIGS. 1 and 3, the fingers 12 of this glove 10 extend up to the first joint 13 of the user's digits. The glove material is thin and flexible, so one or two gloves 10 can be worn around the home, around town, or at work, as desired, without impeding most tasks involving the fingers. The base 14 of the glove 10 may extend past the user's wrist, as shown in FIG. 3, or not, as shown in FIG. 1. The material may be sewn together along a side seam 15, as shown in FIG. 3, forming an upper portion 21A and a lower portion 21B of the glove 10.

As shown in FIGS. 1 and 2, this embodiment of the glove 10 has three layers: an outer protective layer 16, preferably of nylon material or the like, a middle PCM/gel envelope 17, and an inner, conformable layer 18, preferably of nylon or the like. The inner and outer layers 16, 18 are conformable to the skin and the envelope 17. The inner layer 18 is of a nonallergenic material that can be worn comfortably against the skin for hours, as desired.

Continuing with FIGS. 1 and 2, the envelope 17 in the glove 10 holds a gel or viscous fluid carrier 19, such as a urethane gel or an oil, in which phase change material 20 is substantially evenly distributed. It is believed that a gel is optimal for maintaining phase change material distribution and for even heating or cooling. In regard to materials intended to be in contact with the skin, two important parameters for obtaining FDA (Food and Drug Administration) approval are skin irritability and cytotoxicity. The carrier and phase change material are both preferably non-toxic.

For warming gloves and pads herein, the phase change material 20 has a melting point, or phase change temperature, of between about 42 and 65 degrees Centigrade. The item is heated in a microwave or by other suitable heating means, and then placed on the troublesome area of the body. For cooling gloves and pads herein, the phase change material 20 has a melting point, or phase change temperature, of between about 10 and 6 degrees Centigrade. The item is cooled in a freezer, or refrigerator, or by other cooling means, and then placed on the body. For both, the phase change material is one that is capable of being suspended in the gel or fluid carrier herein. Importantly, it is also possible to mix warming and cooling phase change materials in the carrier, obtaining a glove or pad that can be heated or cooled, as desired.

FIGS. 4 through 7 show a mitt 22 according to the present invention. FIG. 4 shows an arthritic hand 23 being inserted into the opening 24 at the base 25 of the mitt 22. A patient with arthritis in her hands could, for example, increase her short term range of motion and grip strength by placing her hands in the heated mitts for about an hour. Warmed mitts 22 are also suitable for use, for example, at a beauty salon for prolonging the warming, softening, soothing effect after dipping the client's hands (or feet) into warm wax. Once the mitts 22 have cooled, the beauty salon client removes her hands from the mitts, and the cooled wax is peeled off.

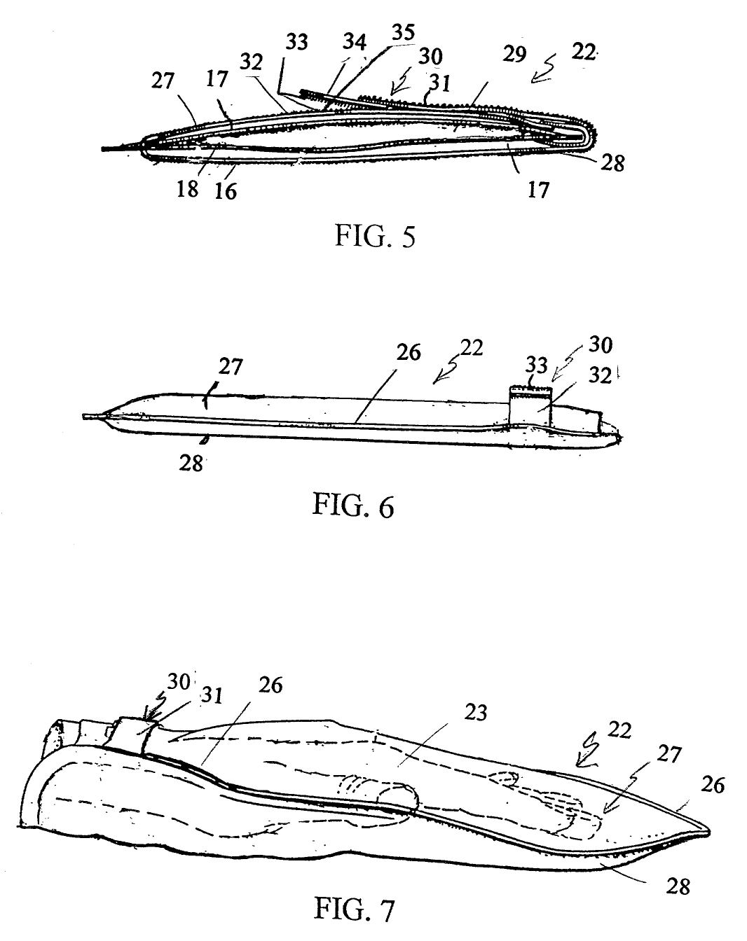

FIGS. 6 and 7 show the mitt 22 from the side. The mitt 22 may be made of similar, arched sections of material which are sewn together after two similar sized PCM-containing envelopes 17 are placed between the inner and outer insulative material layers. The mitt side seam 26, which is shown in FIGS. 6 and 7, joins upper 27 and lower 28 portions of the mitt 22 to one another along the arch. As shown in the FIG. 5 cross-section, the upper portion 27 holds a PCM-containing envelope 17 and the lower portion 28 holds a similar envelope 17. An alternative embodiment may include only one envelope 17 in the upper or lower portion of the mitt. Generally, the mitts 22 herein hold more PCM/carrier than the gloves 10, which are thinner and more flexible than the mitts.

Once the user's hand 23 has been inserted into the central compartment 29 of the mitt 22, a top belt 30 can be fastened over the top of the wrist to retain the warm or cool air around the hand 23 in the mitt 22 (see FIG. 7). The top belt 30 is optional but preferred. It fastens the mitt opening around small or large wrists. As shown in FIGS. 4-6, the top belt 30 is comprised of two complementary strap sections 31, 32. Facing sides of the ends 34, 35 of the two overlapping strap sections 31, 32 hold lock and loop strips 33. Once the hand 23 is inserted into the central compartment 29 of the mitt 22, the strap ends 34, 35 are pulled toward one another and the uppermost strap end 34 is pressed down onto the lock and loop strip 33 on the lowermost strap end 35. The central compartment 29 is bordered by an inner, insulative, comfortable layer 18. The outer, insulative layer 16 covers the outside of the mitt. The mitt 22 accommodates hands of various sizes. The first, glove embodiment 10 may also comprise a similar top belt.

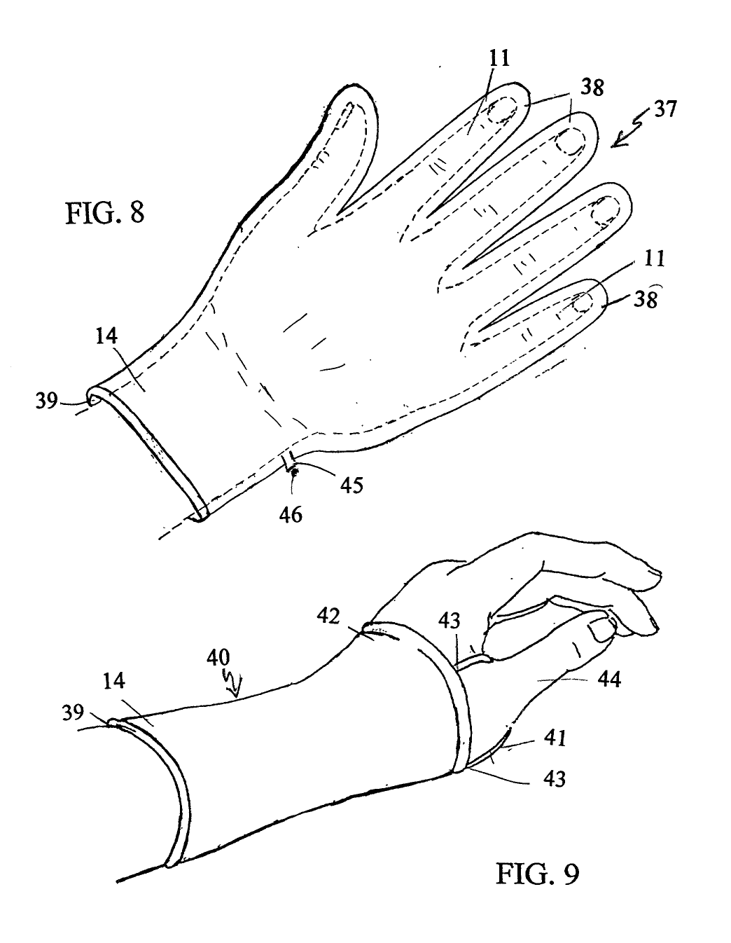

An alternate glove 37 embodiment is illustrated by FIG. 8. Here, the glove 37 has fingertips 38 and a single opening 39 into which the user's fingers 11 are inserted. The base 14 of the glove 37 extends down past the user's wrist. As described herein, the glove 37 comprises a thin, flexible PCM-containing envelope 17 on its top and bottom between the inner and outer insulative layers 16, 18, per FIG. 2. Since it only has one opening 39, which is blocked by the user's arm when the glove 37 is in use, warm or cool air is retained well within the glove 37. Once they are heated in a microwave, a set of these gloves 37 would be particularly useful for a healthy person on a cold winter day. The phase change material in the envelope and the construction of the glove 37 allows heat retention for an extended period of time, e.g., for a morning drive to work.

An arm wrap 40 according to the present invention is illustrated in FIG. 9. This arm wrap 40 can be warmed in a microwave, for example, and worn over a bothersome wrist while the user is performing repetitive hand motions, such as working at a keyboard or on a parts assembly line. Or the arm wrap 40 can be cooled and worn over a sore wrist during a basketball or volleyball game, for example. The PCM-containing envelope 17 (not shown) within the snugly fitting arm wrap 40 is flexible and conforms to the shape of the user's arm, as do the other thin, flexible, stretchy arm wrap materials. The PCM-containing envelope preferably extends the length and breadth of the arm wrap 40, so the warming or cooling effect extends throughout the article 40.

The arm wrap can be narrower, forming a wrist band, or it may be wider than the arm wrap 40 shown in FIG. 9. An arm wrap or band according to the present invention (preferably with a width of about two or three inches) can be fastened snugly around the lower arm to apply pressure and cool temperatures on the tendon leading down to the wrist area. A rectangular or (2 inch) square cooling pad is insertable into a band portion of the arm wrap, and the band portion is wrapped around the widest part of the lower arm so that the pad sits on top of the arm and cools the tendon. This has been found to alleviate the daily pain associated with carpal tunnel syndrome, although it is not a cure.

Continuing with FIG. 9, the arm wrap 40 optionally includes a thumb strap 41 at its uppermost end 42, as shown in FIG. 9 for helping to hold the arm wrap snugly in place. Each end 43 of the thumb strap is attached to the uppermost end 42 of the arm wrap 40, the ends being attached several inches apart. The user's thumb 44 is inserted through the thumb strap 41 as the user's hand is pushed through the opening 39 in the arm wrap 40. The uppermost end 42 of the arm wrap 40 extends up over the user's wrist for warming/cooling and support.

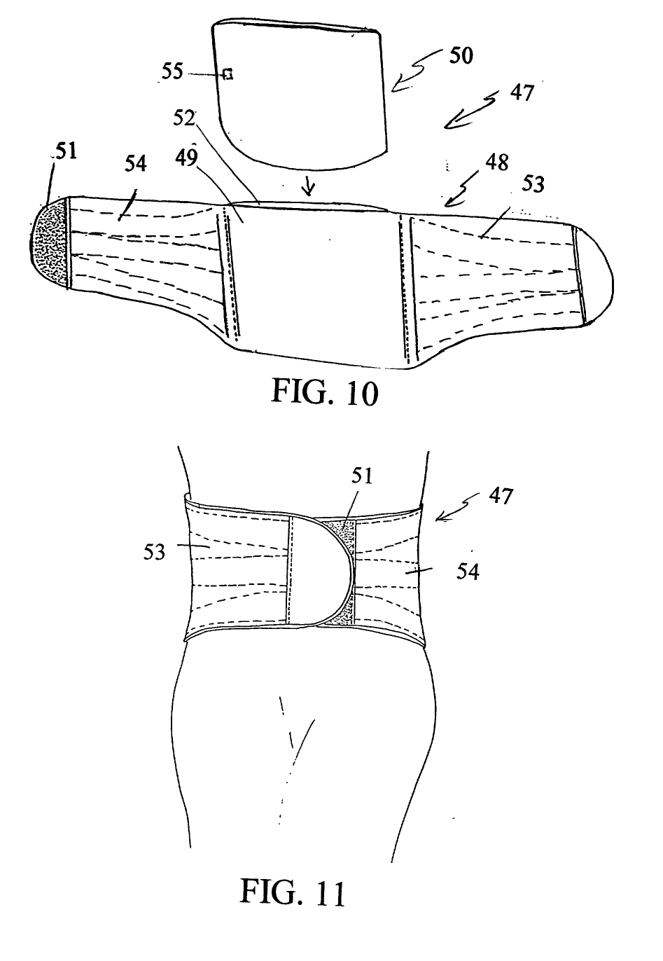

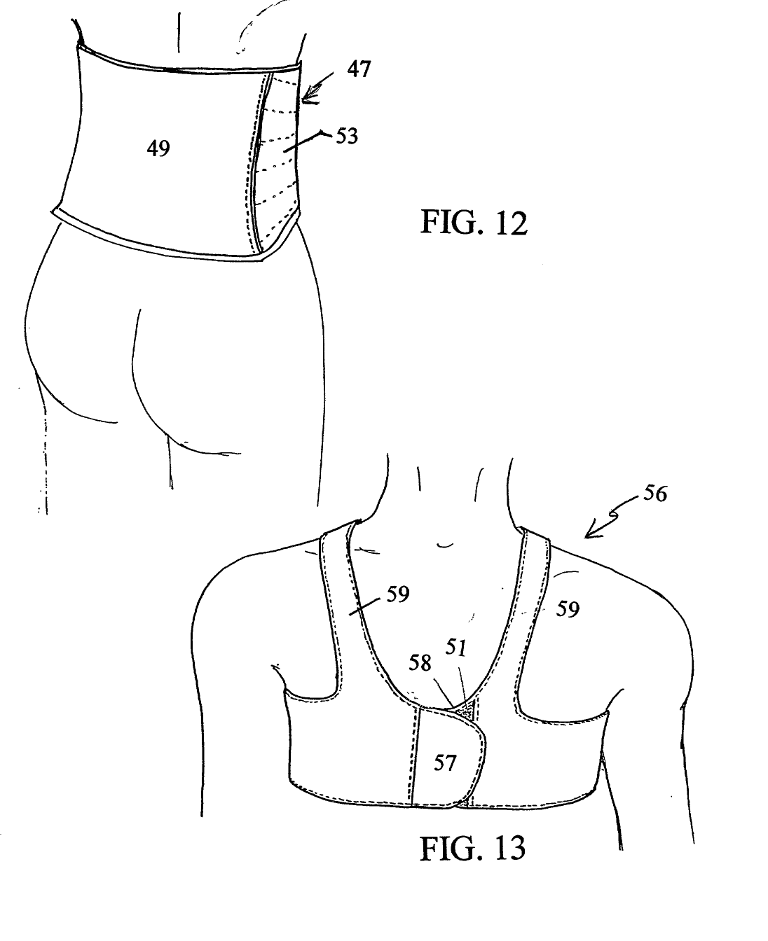

Turning to FIGS. 10-12, a lower back heating/cooling pad device 47 according to the present invention includes a pouch belt 48 having a central pouch 49 for closely accommodating a pad insert 50. FIG. 11 shows a front view of a pad device 47 around the waist of a user, and FIG. 12 shows a rear view. After warming a pad insert 50 in the microwave or by other means, or cooling a pad insert 50 in the freezer or by other means, the pad is inserted into the central pouch 49 in the pouch belt 48, as indicated in FIG. 10. The user then puts on the lower back pad device 47, as shown in FIG. 11, with the pad and pouch over the troublesome lower back area, as shown in FIG. 12. Complementary lock and loop patches 51 are attached to an upper side of one end of the pouch belt and a lower side of the opposite end of the pouch belt 48. The user fastens the pad device 47 around her waist by pressing one end of the belt 48 over the other end.

Lower back pain, which is often of unknown etiology, is a common problem. Localized warmth has been shown to reduce lower back pain, prevent spasms, increase range of motion, and/or reduce the likelihood of re-injury. Though it is primarily intended for applying warmth and pressure over the lumbar vertebrae (in the lower back), the pad device 47 may also function to prevent injuries during lifting. The pad device 47 is comfortable and can be worn during working hours. It can be wide, so that it extends down (over the coccyx or the top of the sciatic nerve, for example) or narrow, depending upon the desired coverage.

Although the pad insert 50 is preferably rectangular or square in shape, as shown in FIG. 10, it can be made in other shapes. The pad insert 50 fits snugly into an opening 52 on the upper side of the similarly sized pouch 49. The other three sides of the pouch 49 are closed. As shown in FIG. 10, two opposite seamed sides of the pouch are connected to a right belt portion 53 and a left belt portion 54, respectively, which are preferably made of an elasticized material. The inner layer of the pad device 47 is a comfortable material, such as cotton material, fleece, or wool. The outer, insulated layer of the pad device 47 is preferably a hollow fiber insulation with a cotton or nylon covering. The surface of the pad device 47 is preferably washable, but if the pad 50 rips or is irreparably soiled, it is inexpensive enough to be discarded and replaced.

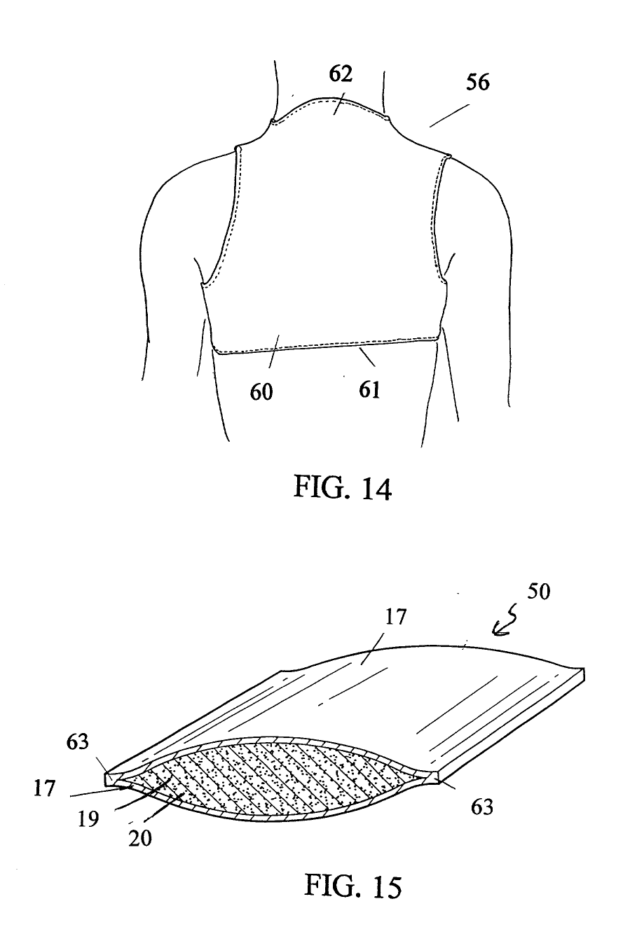

Turning to FIGS. 13 and 14, a heating/cooling pad vest device 56 similarly fastens in the front by means of lock and loop patches 51 at two opposite ends 57 (right), 58 (left). The vest device 56 includes contiguous vest straps 59 that fit over the user's shoulders. Once the pad 50 is warmed or cooled, it is inserted into an opening 61 in the bottom of a similarly sized pouch 60 in the back of the vest device 56. The pad 50 thus warms or cools the user's back in the area of his cervical and thoracic vertebrae. The pad may be long or short, depending on the degree of coverage desired. The top 62 of the vest device 56 is preferably raised as shown in FIG. 14, so that it covers more of the cervical vertebrae (in the neck area). A long, rectangular pad fits up into the top 62 of the vest device 56 for more extensive coverage of the user's cervical and thoracic vertebrae. The inner layer of the vest device 56 is made of a comfortable material, such as cotton material, fleece, or wool. The outer, insulated layer of the vest device 56 is preferably a cotton or nylon material. The vest pad device 56 is preferably washable.

The pad inserts 50 for the lower back device 47 and the vest pad device 56 comprise an envelope 17 containing phase change material 20 distributed in the gel or viscous fluid carrier 19 as described herein. If desired, the pads herein can be heated or cooled and used separately as, for example, cooling pads, operating room positioning pads, etc. Cooling pads are placed, for example, between the knees of a patient lying on his side, or between the patient and the surface on which she is lying. Operating room positioning pads are small pads that are placed wherever they are needed during surgery to reduce pressure points, and hold the patient's body in place. The present pads 50 would have to be used appropriately and carefully in the operating room since the cooled pads 50 are uncommonly cold, particularly where phase change material at the low end of the melting point range is employed, and the heated pads are quite hot, particularly where phase change material at the high end of the melting point range is used. The phase change material melting point for the cold pads 50 ranges between 14.7 (for C12) and 42.6 (for C14) degrees Fahrenheit (−9.6 to 5.9 degrees Centigrade), and the phase change material melting point for the warm pads is between 111.9 (for C22) and 148.5 (for C30) degrees Fahrenheit (44.4 to 64.7 degrees Centigrade). The pads 50 are preferably insulated all around the outside of the envelope 17 to ameliorate the effect of the colder and hotter melting point phase change materials. Insulation also prolongs the cooling and heating effect of the pads.

The viscous fluid or gel carrier 19 in which the phase change material 20 is suspended is contained within a thin, fluid impermeable, conformable envelope 17. For an average sized, rectangular or square shaped envelope of between about two and 12 inches wide and about two and 12 inches long, the envelope preferably contains between about 50 and about 250 grams of the phase change material and between about 30 and 150 grams of the carrier 19. The concentration of phase change material to carrier by weight is preferably between about 1:5 and 5:1, most preferably about 60 PCM:40 carrier. This ratio has generally been found to be of importance because, for most types of phase change materials and carriers, the more phase change material that is added to the carrier beyond a certain minimal level, the stiffer and firmer the mixture becomes. Although in general more phase change material means better and longer warming or cooling, too much phase change material results in an overly bulky, unacceptably rigid envelope. Only a small amount of PCM/carrier is required in each envelope to achieve the desired therapeutic effect, so the devices of the present invention are thin and flexible.

The type of phase change material utilized herein can be varied according to budget, temperature requirements, and length of time that heating or cooling is desired. Although the PCM-carrier mixture is expected to lose heat/cooling after a time, it has been found that a PCM-carrier mixture that remains heated or cooled for longer than about 20 minutes is useful herein.

For the heating applications, the phase change materials used herein preferably melt at a temperature of between about 42 and 65 degrees Centigrade, more preferably between about 44 and 55, degrees Centigrade. The warm phase change material warms the carrier, which warms the urethane film envelope, which warms the insulation, which warms the patient's skin. Warmth in this temperature range helps in short term management of certain types of pain, such as back pain, joint pain related to arthritis or past injury, the ache from a pulled muscle, and menstrual cramps.

Preferably, once the phase change material 20 is suitably heated, the article herein heats the user's skin to between about 37 and 43 degrees Centigrade for between 25 about 20 minutes and three hours. The temperature of the carrier and the phase change material suspended in it must be a few degrees warmer than this in order to drive heat toward the body. Some heat will be lost to the environment once the article of the present invention is placed on the user's body.

Phase change materials are normally classified according to their melting points. Since most phase change materials are not pure, they melt over a range of several degrees of temperature. When they are heated to a temperature within this temperature range, the bulk of the phase change materials within the PCM mixture will melt from a solid to a liquid, and vice versa for cooling. Many variables contribute to the performance of the pads, including the type of phase change material, the mixture, the carrier, whether they are encapsulated, how long they are heated/cooled, the type of article herein, the body temperature of the patient on whom the present article is applied, and their subcutaneous fat layer.

As the warmed phase change material cools, it undergoes a phase change and provides heat to the skin below the envelope. Because this phase change occurs at a specific temperature, the heat can only be delivered at this specific temperature, or slightly less than it, depending upon the thickness of the insulation between the PCM-containing envelope 17 and the covered area of the body. This makes it ideal for temperature-controlled application of heat.

Suitable phase change materials for use in heating applications herein include C22 to C30 alkanes (i.e., alkanes with between about 22 and 30 carbons) or mixtures thereof, and acetate salts, preferably sodium acetate. Preferred alkanes for use herein are docosane (C22), tricosane (C23), tetracosane (C24), pentacosane (C25), hexacosane (C26), octacosane (C28), and triacontane (C30). The melting points for these alkanes range between 44.4 and 64.7 degrees Centigrade, which is between 111.9 and 148.5 degrees Fahrenheit. Combinations of C24 and C26, or C26 and C28 alkanes are useful because they gradually melt over a specific temperature range, depending on which chain lengths are included and the relative amount of each. For example, a 50:50 mixture of C24 and C26 alkanes will melt between about 50.9 and 56.4 degrees Centigrade. Alkanes may also be selected and mixed based on budget constraints, since some of them are much more expensive than others. For example, a 24-carbon paraffin, tetracosane, is expensive. A less expensive alternative is a mixture of C23, C25, C26, and C27 alkanes, which has a peak melting point of 50 degrees C., but the peak of the melting curve for this mixture is broader and less precise than that of tetracosane in that melting actually occurs over a 47 to 51 degree C. range.

Suitable phase change materials for cooling applications herein include C12 to C14 alkanes (i.e., alkanes with between about 12 and 14 carbons), and mixtures thereof. Preferred alkanes for use herein, then, are dodecane (C12), tridecane (C13), and tetradecane (C14). It is believed that cold temperatures in this range are advantageous in preventing or reducing the incidence of involuntary tremors, and in reducing inflammation and edema. The alkanes used in this invention can also be varied according to the degree of cooling desired for the particular part of the body on which the pad (or glove) will be used. Alkanes may be selected according to the degree of cooling necessary to achieve the desired cooling effect.

Phase change material 20 for use herein is preferably microencapsulated so that it remains evenly distributed throughout the carrier 19 even after heating and cooling. This is important because many phase change materials, such as the alkanes, are by themselves poor thermal conductors. The distribution of the phase change material into small, generally spherical capsules with a diameter of between about one and 100 microns significantly enhances heat transfer between the surrounding medium and the phase change material. It also allows for sliding between the individual capsules of phase change material, allowing the mixture to be conformable to the body. (Solid alkane phase change material alone is firm and waxy; it generally maintains a rigid structure until it is melted.) Microencapsulation also prevents interaction, chemical or otherwise, over time between the phase change material and the carrier or envelope material, thus increasing product durability. Any suitable method for encapsulating the phase change material in a protective coating can be utilized. Powder PCM is preferably used because it is believed to have good conductivity due to its higher surface area. The phase change materials are preferably microencapsulated in a thin coating, more preferably a polymer. The coating preferably forms a generally spherical shell around the phase change material with a shell thickness of between about 0.03 and two microns, most preferably about 0.05 micron thick.

A preferred embodiment herein includes:

(a) a fluid-impermeable, flexible, conformable envelope;

(b) a mixture of from about 10 to about 75 weight % of alkanes having a carbon chain length of between 12 and 14 or 22 and 30, and from about 25 to about 90 weight % of a gel or viscous fluid carrier in which the alkanes are substantially evenly distributed, the mixture being sealed within the envelope;

(c) at least one layer of insulation adjacent to a first side of the envelope.

The heating/cooling articles herein are preferably not electrical or otherwise powered. They do not comprise a power source or microprocessor. Prior to using the glove or pad insert, it is placed in a microwave for heating, or a freezer for cooling. Other means of heating, such as submersing it in hot water, or cooling, such as refrigerating it, may be employed. It is important that the glove, pad, etc. not be heated above the recommended temperature range, or frozen, because it could conceivably harm a patient.

Various systems can be used for determining when the glove, pad, etc. is heated or cooled to an appropriate degree. One system includes a small thermal window or slit on the upper surface of the glove 10, 22, 37 or pad insert 50. As shown in FIG. 10, a color-coded window 55 may be included on the glove or pad insert, or on a wrapper around it. The window is visible to the user looking in through the front window of the microwave. The thermal window allows the user to view a thermal indicator placed inside the glove or pad insert, which senses the internal temperature of the carrier and/or envelope. The thermal indicator changes in some visible way, such as a color change, to indicate to the user that the PCM-containing carrier has been heated, or cooled, to the pre-determined temperature. This system is inexpensive because a home microwave can be employed. A color-coded temperature indicator may also be applied directly on the upper or lower portion of the glove or pad insert.

Another way for the user to determine when the glove or pad is properly heated is a pop-up thermometer 45 in the glove 37, or pad insert, as shown in FIG. 8. The lower end of the thermometer rests inside the glove or pad, preferably in, on, or very near the envelope 17 that encloses the PCM-containing carrier 19. When the predetermined temperature is reached, a brightly colored tab 46 on the upper end of the thermometer 45 pops out, signaling to the user that the glove 37, or pad insert 50, is heated and can be removed from the microwave, and used.

Another system for properly heating the glove, pad, etc. herein involves a temperature bar code on a wrapper around the glove or pad. A special microwave outfitted with an identifier, such as a bar code reader or a similar package identifier, identifies the identifier and inputs to the microwave regarding the length of time the microwave is on. The coded, pre-determined time is based on how long it takes for approximately 3/4 of the phase change material to reach its phase change temperature when packaged as identified by the code. That is, the microwave oven is timed to warm the carrier and melt approximately 3/4 of the phase change material. The bar code (or similar) reader reads the temperature bar code and initiates a timed heating cycle at a pre-programmed power setting. The microwave can be programmed for more than one temperature setting or period of time.

Without meaning to be bound by theory, it is believed that leaving about 1/4 of the phase change material unmelted ensures that the glove 10, 22, 37, pad 50, etc. will not be overheated. As long as some phase change material remains in the solid state, the temperature of the envelope 17 will not exceed its phase change temperature. It is believed that it is important not to overheat the envelope 17 because as soon as 100% of the phase change material is melted, the temperature control behavior provided by the phase change is lost. If the phase change material were fully melted, the pad could be heated to temperatures which can harm the skin area and surrounding skin.

The glove, pad, etc. of the present invention preferably do not comprise a power source; since the glove, pad, etc. are pre-heated or pre-cooled, they do not have to be attached to an electrical source or batteries while on the user, nor is control circuitry necessary, in order for the envelope to maintain heat or cold for the required therapeutic period of time. The glove, pad, etc. do not include a microprocessor, EEPROM, or the like.

Referring now to FIG. 15, a cross-section of a pad insert 50 shows an outer urethane film envelope 17 holding carrier 19 in which phase change materials 20 have been distributed. The fluidic PCM/carrier contents are enclosed by the flexible envelope 17, which is then preferably heat sealed along the edges 63. In FIG. 15, the top of the pad 50 is the same as the bottom. In a preferred embodiment, the envelope 17 is urethane film, the carrier 19 is a silicone fluid, and the phase change material 20 is C14 alkane encapsulated into microcapsules having a diameter between about one and 100 microns. These pads 50 can be used, for example, to pack around a patient undergoing a medical procedure that requires cooling of a limb or any other part of the body. Most pads 50 are enclosed by an outer layer of insulation 16, 18.

The envelope 17 is preferably made of a urethane film with a thickness of between about 0.003 and 0.08 inch. The shape and size of the envelope or the pad may be varied according to the article and its intended application. The envelope 17 itself is preferably between about 0.1 and 1.5 inches thick, most preferably 0.5 inch thick for the mitt embodiment. The envelope 17 may include internal divisions. It may, for example, be quilted, with a small amount of phase change material and carrier in each quilt square.

The PCM-containing envelope is preferably surrounded by a conformable thermal insulation layer, which is preferably made of thin sheets of hollow fiber polymer, or any other suitable insulative, cushioning, comfortable, absorbent, conformable material, which provides bulk and softness. The primary function of the insulation layer is to limit the escape of heat or cold from the envelope. The insulation covers the upper and/or lower surface of the envelope 17.

The phase change material is substantially evenly distributed in a carrier 19 which is a gel, such as a urethane gel or a viscous fluid. By “viscous fluid” is meant a fluid with an absolute viscosity between about five and about 100,000 centipoise, most preferably between about five and 10,000 centipoise. As phase change material is added, the carrier stiffens somewhat. Carriers with a lower viscosity are preferable herein because the envelope will be soft and flexible. Urethane gels, silicone fluids, and mineral oils are among the suitable carriers in this regard. It is also believed that inexpensive oils, such as vegetable oil, olive oil, or peanut oil, may also be employed herein, so long as they have a suitably high viscosity and a preservative is included. The carrier and phase change material are both preferably non-toxic, and inter-mixable. The phase change material must maintain distribution within the carrier through a plurality of hot or cold cycles. The viscous, conformable fluid carrier herein should be one that does not foster bacterial or fungal growth when confined in the air- and fluid-impermeable envelope.

It is believed that a viscous fluid is optimal herein for maintaining phase change material distribution and for even cooling and warming. Viscosity is a significant property of the material that is selected to carry the phase change material because it determines, in large part, both the degree of pad conformability and the level to which the phase change material will maintain a relatively even distribution over many use cycles. Viscosity is basically the resistance to change of form exhibited by a fluid. It is a measure of internal friction and is measured as the amount of tangential force exerted by one layer of fluid upon an adjacent layer as it driven across it at a given velocity. The viscosity of the fluid carrier has been found to affect conformability and flexibility of the present invention. The viscosity also affects the tendency for a dispersed powder phase change material to maintain its dispersion. For this reason, very low viscosity fluids, such as water and alcohol, are believed not to be desirable as carriers herein. Polydimethylsiloxane fluid and dimethicone fluid are more preferred for use herein for cooling.

Another consideration for a heating glove or pad herein is the need for the carrier to absorb microwave energy. Urethane gels and mineral oils are among the suitable gel and fluid carriers in this regard. It is also believed that inexpensive oils, such as vegetable oil, olive oil, or peanut oil, may also be employed herein, so long as they have a suitably high viscosity (greater than about 5 centipoise) and high specific heat and a preservative is included. The viscous, conformable fluid herein has a viscosity greater than about five centipoise, and it should have high heat capacity, inhibit bacterial or fungal growth in the air- and fluid-impermeable envelope, and be capable of absorbing large amounts of microwave radiation.

From the foregoing it can be realized that the described heating/cooling articles of the present invention may be easily and conveniently utilized. It is to be understood that any dimensions given herein are illustrative, and are not meant to be limiting.

While preferred embodiments of the invention have been described using specific terms, this description is for illustrative purposes only. It will be apparent to those of ordinary skill in the art that various modifications, substitutions, omissions, and changes may be made without departing from the spirit or scope of the invention, and that such are intended to be within the scope of the present invention as defined by the following claims. It is intended that the doctrine of equivalents be relied upon to determine the fair scope of these claims in connection with any other person's product which fall outside the literal wording of these claims, but which in reality do not materially depart from this invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

### BRIEF LIST OF REFERENCE NUMBERS USED IN THE DRAWINGS

10 first glove

11 user's fingers

12 fingers of first glove

13 first finger joint

14 base of glove

15 seam

16 outer layer

17 envelope

18 inner layer

19 carrier

20 phase change material

21A upper portion of glove

21B lower portion of glove

22 mitt

23 arthritic hand

24 mitt opening

25 base of the mitt

26 mitt side seam

27 upper portion of mitt

28 lower portion of mitt

29 central compartment of mitt

30 top mitt strap

31 right strap section

32 left strap section

33 lock and loop strip

34 the uppermost strap end

35 lowermost strap end

37 second glove embodiment

38 glove fingertips

39 glove opening

40 arm wrap

41 thumb strap

42 uppermost end of arm wrap

43 thumb strap ends

44 user's thumb

45 pop-up thermometer

46 pop-up thermometer tab

47 lower back heating/cooling pad device

48 pouch belt

49 central pouch

50 pad insert

51 lock and loop patches

52 pouch opening

53 right belt portion

54 left belt portion

55 color-coded window on pad

56 heating/cooling pad vest device

57 right front end

58 left front end

59 vest straps

60 rear pouch

61 bottom opening to rear pouch

62 top of the vest pad device

63 edge of envelope

What is claimed is:

1. A heating or cooling pad for reducing pain or swelling, or increasing range of motion, the pad comprising:
(a) phase change material having a melting point of between about 42 and 65 degrees Centigrade, or between about −10 and 6 degrees Centigrade;
(b) a gel or viscous fluid carrier in which the phase change material is substantially evenly distributed; and
(c) a fluid-impermeable, conformable envelope surrounding the phase change material and the carrier.

2. A pad according to claim 1, further comprising at least one conformable insulation layer surrounding the envelope.

3. A pad according to claim 1, wherein the phase change materials are microencapsulated in a protective coating.

4. A pad according to claim 1, which does not comprise a power source or microprocessor.

5. A pad according to claim 1, wherein the envelope is a urethane film.

6. A pad according to claim 5, wherein the phase change material is a C22 to C30 alkane, or a combination thereof.

7. A pad according to claim 6, wherein the phase change material is microencapsulated with a polymer coating, forming generally spherical PCM/polymer microcapsules which range in diameter between about one and about 100 microns.

8. A pad according to claim 4, wherein the carrier is an oil.

9. A pad according to claim 7, wherein the carrier is a silicone fluid.

10. A pad according to claim 7, wherein the carrier is urethane gel.

11. A pad according to claim 5, wherein the phase change material is a C12 to C14 alkane, or a combination thereof.

12. A pad according to claim 1, which is insertable in a corresponding central pouch of a lower back heating or cooling pad device.

13. A pad according to claim 12, wherein the lower back heating or cooling pad device further comprises lock and loop patches attached to facing sides of its opposite ends for fastening the pad device around the waist of a user.

14. A pad according to claim 9, wherein the pad is insertable in a rear pouch of a heating or cooling pad vest device.

15. A glove, mitt or arm wrap for reducing pain or swelling, or increasing range of motion, comprising:
(a) phase change material having a melting point of between about 42 and 65 degrees Centigrade, or between about −10 and 6 degrees Centigrade;
(b) a gel or viscous fluid carrier in which the phase change material is substantially evenly distributed;
(c) a fluid-impermeable, flexible, conformable envelope surrounding the phase change material and the carrier;
(d) a comfortable, conformable inner layer on the inside of the glove or mitt; and
(e) at least one outer, protective, insulative layer on the outside of the glove or mitt;
wherein the envelope is sandwiched between the inner and outer layers on the upper or lower portion of the glove or mitt.

16. A glove, mitt or arm wrap according to claim 15, wherein the phase change material is microencapsulated with a polymer coating, forming generally spherical PCM/polymer microcapsules which range in diameter between about one and about 100 microns.

17. A glove, mitt or arm wrap according to claim 15, which does not comprise a power source or microprocessor.

18. A glove, mitt or arm wrap according to claim 15, wherein the envelope is a urethane film.

19. A glove, mitt or arm wrap according to claim 17, wherein the viscous fluid carrier has a viscosity between about 5 and 10,000 centipoise, and the ratio by weight of fluid carrier to phase change material is between about 1:5 and 5:1.

20. A glove, mitt or arm wrap according to claim 15, wherein the phase change material is a C22 to C30 alkane, or a combination thereof.

21. A glove, mitt or arm wrap according to claim 16, wherein the carrier is a mineral oil.

22. A glove, mitt or arm wrap according to claim 16, wherein the carrier is a silicone fluid or urethane gel.

23. A glove, mitt or arm wrap according to claim 15, wherein the phase change material is a C12 to C14 alkane, or a combination thereof.

24. A glove, mitt or arm wrap for reducing pain or swelling, or increasing range of motion, comprising:
(a) a fluid-impermeable, flexible, conformable envelope;
(b) a mixture of from about 10 to about 75 weight % of alkanes having a carbon chain length of between 12 and 14 or 22 and 30, and from about 25 to about 90 weight % of a gel or viscous fluid carrier in which the alkanes are substantially evenly distributed, the mixture being sealed within the envelope; and
(c) at least one layer of insulation adjacent to a first side of the envelope.
